(19) United States
(12) Patent Application Publication
(10) Pub. No.: US 2020/0360065 A1
(43) Pub. Date: Nov. 19, 2020

(54) ZIP-FIX: A CIRCUMFERENTIAL RAPID FIXATION DEVICES/IMPLANT SYSTEM FOR COMMINUTED OR SIMPLE FRACTURES OF TUBULAR BONE

(71) Applicants: Ellen Wenzel, Washougal, WA (US); Zarko Kajgana, JR., Washougal, WA (US)

(72) Inventors: Ellen Wenzel, Washougal, WA (US); Zarko Kajgana, JR., Washougal, WA (US)

(21) Appl. No.: 16/984,603

(22) Filed: Aug. 4, 2020

(51) Int. Cl. A61B 17/82; A61B 17/88

(52) U.S. Cl. CPC A61B 17/82; A61B 2017/00424; A61B 17/8861

(57) ABSTRACT

An implant system for the rapid fixation of tubular bone fractures utilizing a circumferential, flexible, locking band-like device/implant. The implant can be made of bioresorbable materials and inert non-resorbable materials. The implant is placed circumferentially about a fracture site and with the tip inserted into the slot, a ratcheting pawl engages and secures the fixation in a cerclage type manner. The device is comprised of a locking head with insertion slot with pawl, a strap body with ratcheting teeth, and a transition end which is attached to a pop-off needle via suture or extruded monofilament, or an extruded needle-like expansion, or free for use with free needle and suture.

### FEDERALLY SPONSORED RESEARCH

Not applicable

### BACKGROUND OF THE INVENTION

Surgeons practicing within the various orthopaedic specialties can often find themselves challenged when selecting solely standard metal based or bioresorbable internal fixation for fractures being surgically repaired that do not follow textbook constructs; this is particularly true in the case of comminuted fractures. This can lead to increased operating room times, increased time to heal or delays in healing, and/or inadequate fracture repair resulting in non-anatomical reduction or malunion, as well as the potential for increased costs of implants. In addition, complex fracture reduction implant sets can be costly and may require additional staff for their use making these systems and devices/implants only readily available only in areas that have access to these added resources.

The need arises for a fracture repair devices/implants that can be rapidly deployed in the operative setting to stabilize complex fracture patterns with ease without the need for complex or expensive instrumentation which has the ability to reduce operative and anesthesia time for the patient by more rapidly reducing and stabilizing fractures with improved anatomical reduction, and which can deployed to a larger segment of the population within a variety of medical delivery settings to improve postoperative outcomes in orthopaedic fracture repair.

We present a novel fixation system of devices/implants that can achieve rapid fixation of comminuted fractures through use of these implants in an independent fashion or as an adjunct to standard metallic or bioresorbable fixation methods. This fixation system can be utilized within the human medical fields across civilian, military, and mission based fracture repair needs. In addition, it can be appropriately used across species within veterinary medicine. Further, it has the potential to be packaged in convenient sterile single device units facilitating the ease of deployment in a variety of practice settings.

### SUMMARY OF THE INVENTION

Zip-Fix is a fracture fixation system of devices/implants that can be used independently or in conjunction with standard fixation types for the reduction and fixation of fractures of the osseous structures with extensive application within both human and veterinary medicine. The device/implant is attached to a needle, with or without a tie, which can be removed for passage without the needle or removed after the passage of the device about the bone, which can be applied to the fracture site with increased speed and efficacy. The device/implant can be manufactured from materials already commonly used in fracture fixation applications, including: bioresorbable materials; non-bioresorbable materials that are inert; and, inert malleable metals. Zip-Fix can be coated with antimicrobial and/or radiopaque coatings, or not, depending on the application and material. Zip-Fix can be manufactured in a variety of sizes, lengths, and widths to accommodate a variety of bone diameters and fracture configurations.

### Advantages

There are several advantages to this novel device/implant for fixation. The primary advantage being the speed with which, particularly comminuted, fractures can be stabilized when the bone construct cannot support standard screw or pin style fixation. The device has the potential to be individually packaged for ease of access and use in a variety of medical settings, both human and veterinary, as well as being more convenient for field hospital setting usage. Because the device/implant can be made from bioresorbable materials, the device resorbs without the need for future removal; and, when made from inert non-metallic materials, the risk of unknown metallurgical allergy is averted.

### BRIEF DESCRIPTION OF THE DRAWINGS

Having described the invention in general terminology, reference will now be made to the accompanying drawings and embodiments.

FIG. 1A Diagram illustrating the front plane view of the Zip-Fix showing the cogged, or ratcheting side of the strap, with one end demonstrating a pop-off needle attached to the device/implant via suture and opposite end demonstrating the locking head mechanism.

FIG. 1B Diagram illustrating the cross-sectional view showing the smooth reverse side of the Zip-Fix with one end demonstrating a pop-off needle attached to the device/implant via suture and opposite end demonstrating the locking head mechanism.

FIG. 2A Diagram illustrating the front plane view of the Zip-Fix showing the cogged, or ratcheting side of the strap, with one end demonstrating a pop-off needle attached to the device/implant via suture and opposite end demonstrating the locking head mechanism.

FIG. 2B Diagram illustrating the cross-sectional view showing the alternate textured reverse side of the Zip-Fix with one end demonstrating a pop-off needle attached to the device/implant via suture and opposite end demonstrating the locking head mechanism.

FIG. 3A Diagram illustrating the front plane view of the Zip-Fix showing the cogged, or ratcheting side of the strap, with one end demonstrating a pop-off needle attached to the device/implant via a monofilament and opposite end demonstrating the locking head mechanism.

FIG. 3B Diagram illustrating the cross-sectional view showing the smooth reverse side of the Zip-Fix with one end demonstrating a pop-off needle attached to the device/implant via a monofilament and opposite end demonstrating the locking head mechanism.

FIG. 4A Diagram illustrating the front plane view of the Zip-Fix showing the cogged, or ratcheting side of the strap, with one end demonstrating a pop-off needle attached to the device/implant via a monofilament and opposite end demonstrating the locking head mechanism.

FIG. 4B Diagram illustrating the cross-sectional view showing the alternate textured reverse side of the Zip-Fix with one end demonstrating a pop-off needle attached to the device/implant via a monofilament and opposite end demonstrating the locking head mechanism.

FIG. 5A Diagram illustrating the front plane view of the Zip-Fix showing the cogged, or ratcheting side of the strap, with on one end showing an extruded needle-like expansion and opposite end demonstrating the locking head mechanism.

FIG. 5B Diagram illustrating the cross-sectional view showing the smooth reverse side of the Zip-Fix with one end showing an extruded needle-like expansion and opposite end demonstrating the locking head mechanism.

FIG. 6A Diagram illustrating the front plane view of the Zip-Fix showing the cogged, or ratcheting side of the strap, with on one end showing an extruded needle-like expansion and opposite end demonstrating the locking head mechanism.

FIG. 6B Diagram illustrating the cross-sectional view showing the alternate textured reverse side of the Zip-Fix with one end showing an extruded needle-like expansion and opposite end demonstrating the locking head mechanism.

FIG. 7A Diagram illustrating the front plane view of the Zip-Fix illustrating the cogged, or ratcheting side of the strap, with an open hole/loop for attachment and use with a free suture and needle, and opposite end demonstrating the locking head mechanism.

FIG. 7B Diagram illustrating the smooth reverse side of the Zip-Fix with an open hole/loop for attachment of free suture with needle and opposite end demonstrating the locking head mechanism.

FIG. 8A Diagram illustrating the front plane view of the Zip-Fix illustrating the cogged, or ratcheting side of the strap, with an open hole/loop for attachment and use with a free suture and needle, and opposite end demonstrating the locking head mechanism.

FIG. 8B Diagram illustrating the smooth reverse side of the Zip-Fix with an open hole/loop for attachment of free suture with needle and opposite end demonstrating the locking head mechanism.

FIG. 9A Diagram illustrating the cross-sectional view of a Zip-fix passing device for passing of the device/implant insertion about the osseous structures; this can be constructed from surgical stainless steel, titanium, plastics, or a combination, packaged for single use.

FIG. 9B Diagram illustrating the top plane view of a Zip-fix passing device for passing of the device/implant insertion about the osseous structures; this can be constructed from surgical stainless steel, titanium, plastics, or a combination, packaged for single use.

### DRAWINGS—REFERENCE NUMERALS

| | |
|---|---|
| 1 | transition section or tail end between the |
| | device strap or body and the insertion end |
| 2 | locking head mechanism (non-releasable or |
| | releasable) |
| 3 | uni-directional ratcheting pawl |
| 4 | insertion slot |
| 5 | ratcheting teeth on strap or body component |
| 6 | textured reverse of the strap or body |
| 7 | suture |
| 8 | ratcheting strap or body with ratcheting |
| | teeth (5) |
| 9 | needle |
| 10 | suture attachment hole |
| 11 | crimped needle |
| 12 | monofilament extrusion |
| 13 | needle-like extrusion |
| 20 | Handle |
| 21 | Insertion guide tunnel body |
| 22 | Insertion guide tunnel openings |

### DETAILED DESCRIPTION OF VARIOUS EMBODIMENTS OF THE INVENTION

The present disclosure is a solution for a rapidly deployed fracture reduction and fixation device manufactured of either bioresorbable material or inert non-bioresorbable material and which consists of a fastener end with insertion slot on one end, as either a non-releasable or a releasable style head, with an integrated cog rack opposite a tail end separated by an elongated ratcheting or cog strap. When the tail end is engaged into the fastener end, forming a loop about the osseous structure being fixated, the pawl teeth engage the cogs, locking the device in place. A releasable style head option, allows for release and repositioning and re-engagement of the device/implant.

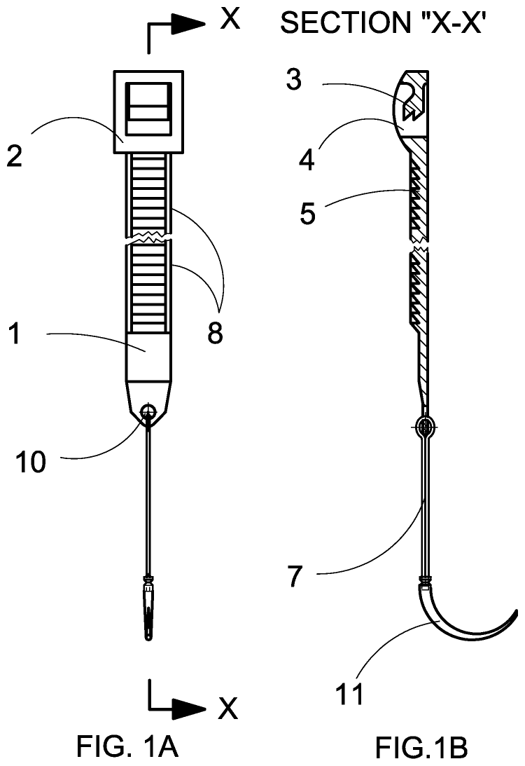

The device/implant of all embodiments demonstrated in FIGS. 1 through 8 can be fabricated from bioresorbable flexible materials, but can also be fabricated from non-bioresorbable inert materials or malleable surgical metals to produce a flexible device/implant.

One embodiment of the device/implant is illustrated in FIGS. 1A (top plane view) and 1B (cross-sectional view). The device/implant is composed of a central strap or body component (8) on which there are ratcheting teeth (5). On one end of the strap body (5) is a locking head (2) with an insertion slot (4), inside which there is a unidirectional ratcheting pawl (3). The pawl within the head can be releasable or non-releasable. On the opposite end of the central strap body (5) the tail end transition (1) has a hole (10) to which suture type material is attached (7) via crimps and onto which a needle (11) is attached. FIG. 1B of the embodiment shows the reverse of the strap (8) with a smooth back for lowest profile of the device/implant against the osseous structure(s) after placement or insertion. The needle and suture may be removed after placement of the device/implant or prior if placement necessitates it. The device is intended to be placed with the smooth side of the device against the osseous structure, being looped around in a circumferential manner, and after which the tail end is inserted into the insertion head, tightened, and locked. The excess material outside of the looped and locked construct is then trimmed to be flush with the locking head.

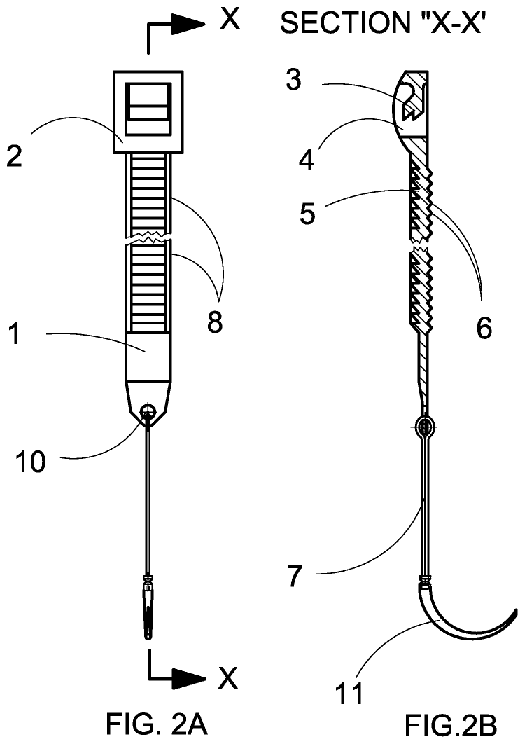

The second embodiment of the device/implant is illustrated in FIGS. 2A (top plane view) and 2B (cross-sectional view). The device/implant is composed of a central strap or body component (8) on which there are ratcheting teeth (5). On one end of the strap body (5) is a locking head (2) with an insertion slot (4), inside which there is a unidirectional ratcheting pawl (3). The pawl within the head can be releasable or non-releasable. On the opposite end of the central strap body (5) the tail end transition (1) has a hole (10) to which suture type material is attached (7) via crimps and onto which a needle (11) is attached. FIG. 1B of the embodiment shows the reverse of the strap body (8) with texture (6) on the reverse of the strap body for gripping the osseous structures to prevent slippage of the device/implant on the osseous structure(s) after placement. The needle and suture may be removed after placement of the device/implant or prior if placement necessitates it. The device is intended to be placed with the textured side of the device against the osseous structure, being looped around in a circumferential manner, and after which the tail end is inserted into the insertion head, tightened, and locked. The excess material outside of the looped and locked construct is then trimmed to be flush with the locking head.

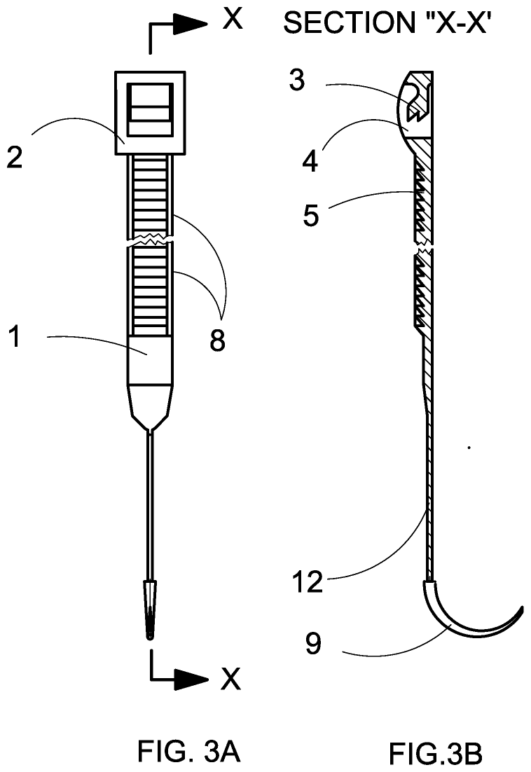

The third embodiment of the device/implant is illustrated in FIGS. 3A (top plane view), 3B (cross-sectional view). The device/implant is illustrated showing a central strap or body component (8) on which there are ratcheting teeth (5). On one end of the strap body is a locking head (2) with an insertion slot (4), inside which there is a unidirectional ratcheting pawl (3). The pawl within the head can be releasable or non-releasable. On the opposite end of the central strap (8) the tail end (1) transitions into an extruded monofilament (12) onto which a needle (9) is attached. FIG. 3B of the embodiment shows the reverse of the strap body (8) with a smooth back for lowest profile of the device/implant against the osseous structure(s) after placement or insertion. The needle may be removed and the monofilament trimmed after placement of the device/implant or prior if placement necessitates it. The device is intended to be placed with the smooth side of the device against the osseous structure, being looped around in a circumferential manner, and after which the tail end is inserted into the insertion head, tightened, and locked. The excess material outside of the looped and locked construct is then trimmed to be flush with the locking head.

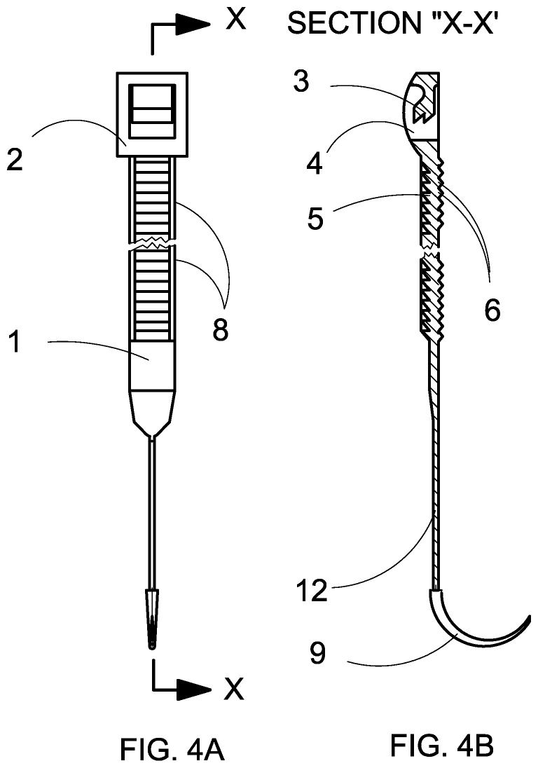

In the fourth embodiment of the device/implant as show in FIGS. 4A (top plane view) and 4B (cross-sectional view), the device/implant is shown composed of a central strap or body component (8) on which there are ratcheting teeth (5). On one end of the strap body (5) is a locking head (2) with an insertion slot (4), inside which there is a unidirectional ratcheting pawl (3). The pawl within the head can be releasable or non-releasable. On the opposite end of the central strap body (5) the tail end (1) transitions into an extruded monofilament (12) onto which a needle (9) is attached. FIG. 4B of the embodiment shows the reverse of the strap body (8) with texture (6) on the reverse of the strap body for gripping the osseous structures to prevent slippage of the device/implant on the osseous structure(s) after placement. The needle may be removed and the monofilament trimmed after placement of the device/implant or prior if placement necessitates it. The device is intended to be placed with the textured side of the device against the osseous structure, being looped around in a circumferential manner, and after which the tail end is inserted into the insertion head, tightened, and locked. The excess material outside of the looped and locked construct is then trimmed to be flush with the locking head.

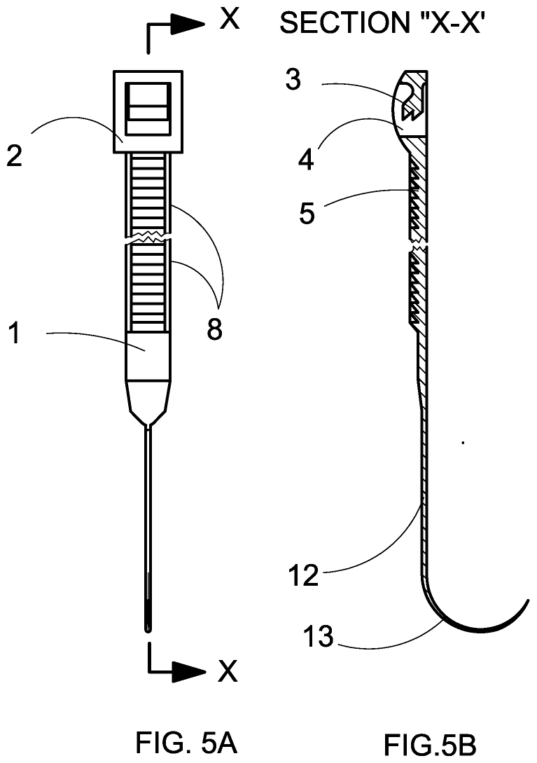

The fifth embodiment of the device/implant is illustrated in FIGS. 5A (top plane view) and 5B (cross-sectional view). The device/implant is composed of a central strap or body component (8) on which there are ratcheting teeth (5). On one end of the strap body (5) is a locking head (2) with an insertion slot (4), inside which there is a unidirectional ratcheting pawl (3). The pawl within the head can be releasable or non-releasable. On the opposite end of the central strap body (5) the tail end transition (1) taper into a monofilament extrusion (12), which terminates in a needle shape (13); this is a uni-body construction. FIG. 5B of the embodiment shows the reverse of the strap (8) with a smooth back for lowest profile of the device/implant against the osseous structure(s) after placement or insertion. The extruded needle-like may be cut and the monofilament trimmed after placement of the device/implant or prior if placement necessitates it. The device is intended to be placed with the smooth side of the device against the osseous structure, being looped around in a circumferential manner, and after which the tail end is inserted into the insertion head, tightened, and locked. The excess material outside of the looped and locked construct is then trimmed to be flush with the locking head.

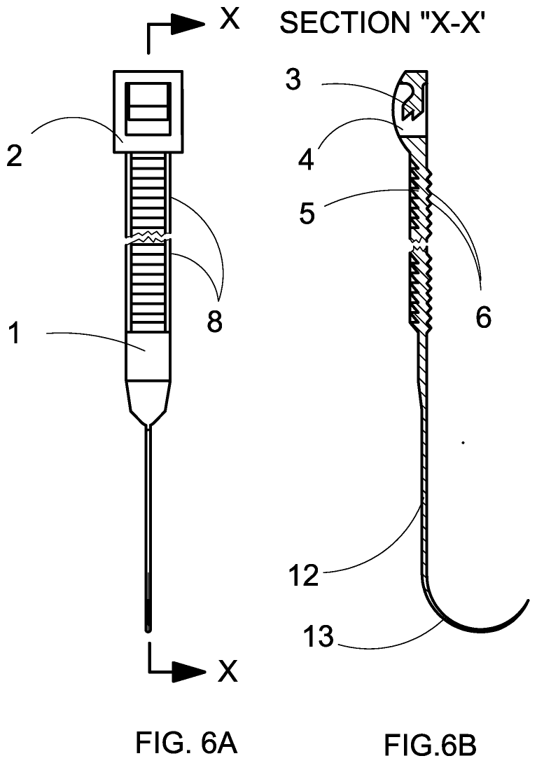

The sixth embodiment of the device/implant is illustrated in FIGS. 6A (top plane view) and 6B (cross-sectional view). The device/implant is composed of a central strap or body component (8) on which there are ratcheting teeth (5). On one end of the strap body (5) is a locking head (2) with an insertion slot (4), inside which there is a unidirectional ratcheting pawl (3). The pawl within the head can be releasable or non-releasable. On the opposite end of the central strap body (5) the tail end transition (1) tapers into a monofilament extrusion (12), which terminates in a needle shape (13); this is a uni-body construction. FIG. 6B of the embodiment shows the reverse of the strap body (8) with texture (6) on the reverse of the strap body for gripping the osseous structures to prevent slippage of the device/implant on the osseous structure(s) after placement. The extruded needle-like may be cut and the monofilament trimmed after placement of the device/implant or prior if placement necessitates it. The device is intended to be placed with the textured side of the device against the osseous structure, being looped around in a circumferential manner, and after which the tail end is inserted into the insertion head, tightened, and locked. The excess material outside of the looped and locked construct is then trimmed to be flush with the locking head.

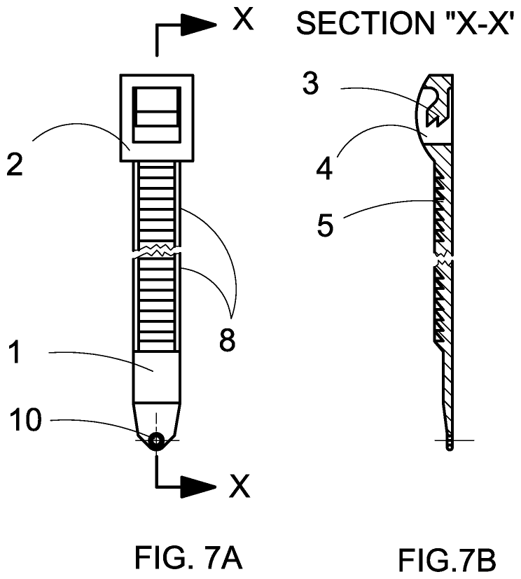

In the seventh embodiment the device/implant is as illustrated in FIGS. 7A (top plane view) and 7B (cross-sectional view). The device/implant is illustrated showing a central strap or body component (8) on which there are ratcheting teeth (5). On one end of the strap body (8) is a locking head (2) with an insertion slot (4), inside which there is a uni-directional ratcheting pawl (3). The pawl within the head can be releasable or non-releasable. On the opposite end of the central strap body (5) the tail end transition (1) has a hole (10) to which any available free needle and sutures may be passed through. FIG. 7B of the embodiment shows the reverse of the strap (8) with a smooth back for lowest profile of the device/implant against the osseous structure(s) after placement or insertion. The device is intended to be placed with the smooth side of the device against the osseous structure, being looped around in a circumferential manner, and after which the tail end is inserted into the insertion head, tightened, and locked. The excess material outside of the looped and locked construct is then trimmed to be flush with the locking head.

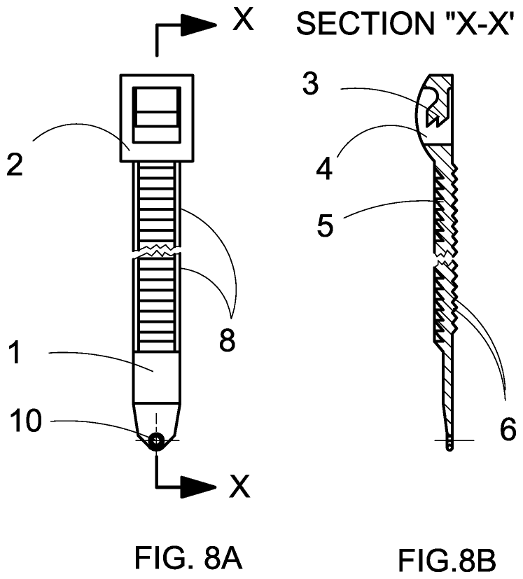

The eight embodiment the device/implant is illustrated in FIGS. 8A (top plane view) and 8B (cross-sectional view). The device/implant is illustrated showing a central strap or body component (8) on which there are ratcheting teeth (5). On one end of the strap body (8) is a locking head (2) with an insertion slot (4), inside which there is a uni-directional ratcheting pawl (3). The pawl within the head can be releasable or non-releasable. On the opposite end of the central strap body (5) the tail end transition (1) has a hole (10) to which any available free needle and sutures may be passed through.

FIG. 7B of the embodiment shows the reverse of the strap (8) with texture (6) on the reverse of the strap body for gripping the osseous structures to prevent slippage of the device/implant on the osseous structure(s) after placement. The device is intended to be placed with the textured side of the device against the osseous structure, being looped around in a circumferential manner, and after which the tail end is inserted into the insertion head, tightened, and locked. The excess material outside of the looped and locked construct is then trimmed to be flush with the locking head.

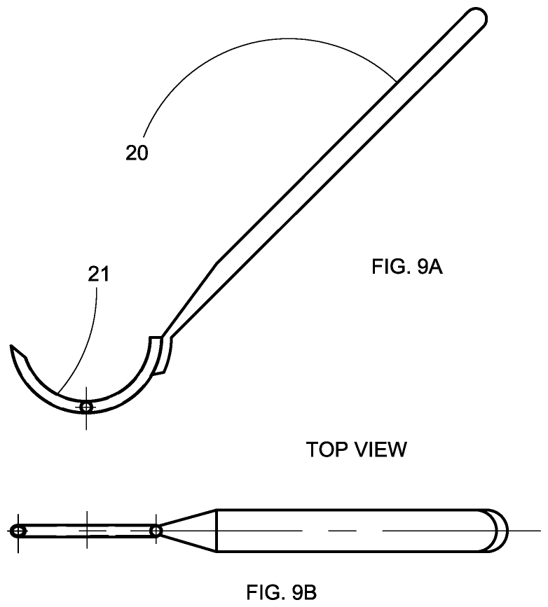

The final, or ninth embodiment, of the device/implant system is for an insertion guide or device and is illustrated in FIGS. 9A and 9B. The device is compromised of a handle (20) and an insertion slot or tube (21), which is noted to have open ends (22). The handle may be comprised of metallic or materials that can be autoclaved or sterilized, and can be packaged for single use or manufactured for repeated sterilization and multiple uses. The open holes (22) on either side of the slot may be round or oblong and manufactured in multiple sizes and/or diameters. The end farthest from the handle, or tip, is manufactured with a long scoop end to aid in tissue dissection. The insertion guide is used in the following manner: the tip end is introduced into the surgical site and then advanced deep to the tubular bone being fixated. Pressure is then employed to advance the device in a semicircular motion about this bone. Once the device tip is visible on the opposite side of the structure for which a Zip-Fix will be used for fixation, the chosen Zip-Fix fixation is guided through the tip end, until it is visible on the handle side of the insertion tube. If needed, the surgeon can remove the needle, extruded needle like end, or the like, in order to facilitate guidance of the device through the slot. Once the Zip-Fix implant has been advanced through the slot adequately, the insertion guide will be removed by pulling in the direction opposite to its original insertion.

In use, each of the embodiments is inserted around a tubular bone with either the smooth reverse or the textured reverse against the osseous structure. The fracture can be reduced and temporarily fixated (i.e. pinned) if desired, prior to placement of the Zip-fix device. Once adequately reduced, and with the Zip-Fix in proper position in a circumferential manner about the tubular bone, the needle or needle-like ends are cut from the device. If a free needle is used, it and suture are pulled out. Next, the tail end of the Zip-Fix implant is then placed through the insertion slot and then tightened, engaging the pawl on the ratcheting teeth. Once the appropriate amount of tightness has been applied to the fracture site by the implant, the free end is cut flush with the locking head. If needed, multiple Zip-Fix devices/implants can be used on a single fracture.

It is understood that the figures and embodiments are not drawn to scale and that the relationship between objects may not be to scale to one another. The figures are intended to give clarity to the structures of the objects and therefore may be exaggerated in some drawings in order to highlight a particular feature. In addition, although the various embodiments and illustrations are included, any number of changes may be made to the various embodiments without departing from the original scope of the invention. Optional features of the device/implant system may be included in some embodiments, and not in others but this is not to be interpreted as to limit the scope of the invention as set forth in the claims.

### CONCLUSION, RAMIFICATIONS, AND SCOPE

The normal fixation of fractures requires the use of implanted devices, particularly screw and/or pin fixation with or without plating. This standard fixation methodology makes reduction of fractures that do no follow the textbook fracture patterning to be repaired with ease. We present a novel device/implant for the fixation of fractures, particularly the comminuted types of fractures in tubular bones, which can be easily and rapidly deployed for circumferential fixation. This new device/implant can be used in both human and veterinary medicine and can be deployed in a variety of medical settings with great ease, without the need for expensive instrumentation sets nor the need for manufacturer representation for placement of complicated devices/implants.

What is claimed is:

1. A surgical device/implant for the circumferential fixation of fractures, manufactured from bioresorbable material or inert non-resorbable materials, whether uncoated or coated in antimicrobial and/or radiopaque coatings, and manufactured in various widths and lengths to accommodate varying anatomical diameters and structures, and comprising:
(a) a central strap or body component on which there are ratcheting teeth on one side with a smooth reverse; and,
(b) on one end of said central strap body, there is a locking head with an insertion slot, within which there is a unidirectional pawl; and,
(c) on the end opposite said locking had on said central strap body there is tail end transition which is inserted through said locking head after being passed about the osseous structure, and tightened to achieve fixation.

2. The surgical device/implant of claim 1, wherein attached to said tail end transition, there is suture type material is attached for passing the device about the osseous structure being fixated.

3. The surgical device/implant of claim 1, wherein said tail end transition continues as an extruded monofilament.

4. The surgical device/implant of claim 1, wherein said tail end transition continues as a monofilament extrusion which tapers into a needle-like expansion in a uni-body design.

5. The surgical device/implant of claim 1, wherein said tail end transition comprises a hole only for use with free suture and needle.

6. The surgical device/implant of claim 3, wherein a pop-off needle is attached to said monofilament end.

7. The surgical device/implant of claim 1, wherein the non-ratcheting side on said central strap body is textured.

8. The surgical device/implant of claim 1, wherein ratcheting pawl of said locking head may be releasable for adjustment.

9. The surgical device/implant of claim 1, wherein said strap body may be manufactured in a mesh-like pattern.

10. An insertion device for the insert of the device/implant of claim 1 comprising a handle and a tubular directing end and which can be manufactured and packaged for single-use or resterilization.
